(19) United States
(12) Patent Application Publication
(10) Pub. No.: US 2015/0032019 A1
(43) Pub. Date: Jan. 29, 2015

(54) END-TIDAL GAS MONITORING APPARATUS

(71) Applicants: Jaron Acker, Madison, WI (US); David Christensen, Cambridge, WI (US); John C. Falligant, Edgerton, WI (US); Michael A. Insko, Lake Stevens, WA (US); John Klaus, Cottage Grove, WI (US); Federick J. Montgomery, Sun Prairie, WI (US); Christopher Toombs, Sammamish, WA (US)

(72) Inventors: Jaron Acker, Madison, WI (US); David Christensen, Cambridge, WI (US); John C. Falligant, Edgerton, WI (US); Michael A. Insko, Lake Stevens, WA (US); John Klaus, Cottage Grove, WI (US); Federick J. Montgomery, Sun Prairie, WI (US); Christopher Toombs, Sammamish, WA (US)

(73) Assignee: Fred Hutchinson Cancer Research Center, Seattle, WA (US)

(21) Appl. No.: 14/129,760

(22) PCT Filed: Jun. 27, 2012
(86) PCT No.: PCT/US2012/044348

§ 371 Date: Aug. 18, 2014

Related U.S. Application Data
(60) Provisional application No. 61/501,844, filed on Jun. 28, 2011.

(51) Int. Cl. A61B 5/083; A61B 5/08; A61B 5/00; A61B 5/097

(52) U.S. Cl. CPC A61B 5/0836; A61B 5/097; A61B 5/082; A61B 5/746
USPC 600/532

(57) ABSTRACT

A non-invasive monitoring apparatus for end-tidal gas concentrations, and a method of use thereof, is described for the detection of endogenous gas concentrations, including respiratory gases, in exhaled breath.

### TECHNICAL FIELD

The present invention relates to non-invasive monitoring of end-tidal gas concentrations in expired air, and, more particularly, to a method and apparatus for the detection of end-tidal gas concentrations, including hydrogen sulfide, carbon dioxide, carbon monoxide, nitric oxide and other respiratory gases, via detection of concentrations of such agents in exhaled breath.

### BACKGROUND

Hydrogen sulfide (H₂S) is a gaseous biological mediator with functions as a signaling molecule and potential therapeutic agent under physiological conditions. H₂S also appears to be a mediator of key biological functions including life span and survivability under severely hypoxic conditions. Emerging studies indicate the therapeutic potential of H₂S in a variety of cardiovascular diseases and in critical illness.

Augmentation of endogenous hydrogen sulfide concentrations by parenteral sulfide administration can be used for the delivery of H₂S to the tissues. Recent studies have also shown that in many pathophysiological conditions, parenteral sulfide administration may be of therapeutic benefit. For instance, parenteral sulfide administration has been shown to be of therapeutic benefit in various experimental models including myocardial infarction, acute respiratory distress syndrome, liver ischaemia and reperfusion, and various forms of inflammation.

However, precise measurement of H₂S concentration in biological fluids is difficult because H₂S is evanescent and reactive. Thus, prior to the claimed invention, the determination of sulfide concentration in blood has relied on assays which require a complicated chemical derivitization procedure.

Nitric oxide (NO) is a low molecular weight inorganic gas that has also been established as a biological mediator. Carbon monoxide (CO) is formed in mammalian tissues together with biliverdin by inducible and/or constitutive forms of haem oxygenase, and has been implicated as a signaling molecule, not only in the central nervous system (especially olfactory pathways) and cardiovascular system but also in respiratory, gastrointestinal, endocrine and reproductive functions. Hydrogen sulfide, nitric oxide and carbon monoxide may also have vasodilator, anti-inflammatory and cytoprotective effects at low concentrations in contrast to causing cellular injury at higher concentrations.

Normally, the exhaled breath of a person contains water vapor, carbon dioxide, oxygen, and nitrogen, and trace concentrations of carbon monoxide, hydrogen and argon, all of which are odorless. Other gases that may be present in exhaled breath include, but are not limited to, hydrogen sulfide, nitric oxide, methyl mercaptan, dimethyl disulfide, indole and others.

Generally, the exhalation gas stream comprises sequences or stages. At the beginning of an exhalation cycle, there is an initial stage the exhaled gases originates from an anatomic location (deadspace) of the respiratory system which does not participate in physiologic gas exchange. In other words, the gas from the initial stage originates from a “deadspace” of air filling the mouth and upper respiratory tracts. This is followed by a plateau stage. Early in the plateau stage, the gas is a mixture of deadspace and metabolically active gases. The last portion of the exhaled breath is comprised of air almost exclusively arising from deep lung, so-called alveolar gas. This gas, which comes from the alveoli, is referred to as end-tidal gas, the composition of which is highly indicative of gas exchange and equilibration occurring between air in the alveolar sac and blood in capillaries of the pulmonary circulation.

Exhaled H₂S represents a detectable route of elimination of endogenously produced sulfide. In addition, exhaled H₂S can also be used to detect augmented sulfide levels after parenteral administration of a sulfide formulation. Recent studies in a rat and human models show that exhalation of H₂S gas can occur when a sulfide formulation or other H₂S donors are administered intravenously.

There is a need in the art for a method and apparatus for non-invasive monitoring of end-tidal gas concentration in blood, and, more particularly, to a method and apparatus for the detection, quantification and trending of end-tidal gas concentration, including hydrogen sulfide, nitric oxide, carbon monoxide, carbon dioxide and other respiratory gases, utilizing the exhaled breath of a patient. There is also a need for an apparatus capable of measuring end-tidal gas concentrations in the exhaled breath of human patients subjected to increasing doses of medications in human safety and tolerability studies. Specifically, there is a need for an apparatus capable of measuring H₂S concentrations in the exhaled breath of human patients subjected to increasing doses sodium sulfide in human safety and tolerability studies, e.g., as required by the U.S. Food and Drug Administration.

### SUMMARY OF THE INVENTION

An embodiment of the present invention provides an end-tidal gas monitoring apparatus for monitoring gas in the exhaled breath of a mammal comprising a gas conduit configured for fluid communication with the exhaled breath of a mammal; a diverter valve in fluid communication with the gas conduit, wherein the diverter valve controls gas flow to a gas sensor downstream of the diverter valve; a CO₂ sensor upstream of the diverter valve in communication with a controller which determines CO₂ levels in the exhaled breath of a mammal to determine when the diverter valve should direct gas flow to the gas sensor; and a recirculation loop downstream of the diverter valve to provide a continuous gas flow to the gas sensor. According to certain embodiments of the invention, the gas sensor is a hydrogen sulfide gas sensor, carbon monoxide gas sensor, carbon dioxide gas sensor, hydrogen gas sensor, nitric oxide gas sensor, or nitrogen dioxide gas sensor.

According to certain embodiments of the invention, the end-tidal gas monitoring apparatus for monitoring gas in the exhaled breath of a mammal further comprises a computer operably coupled to the gas sensor component; a memory component operably coupled to the computer; a database stored within the memory component. According to certain embodiments of the invention, the computer is configured to calculate and collect cumulative data on an amount of exhaled gas by the mammal. According to certain embodiments of the invention, the computer is capable of providing information that alerts a user of the computer of a significant deviation of exhaled gas concentrations from predetermined exhaled gas levels. According to certain embodiments of the invention, the exhaled gas concentration is end-tidal hydrogen sulfide concentration, end-tidal carbon monoxide concentration, end-tidal carbon dioxide concentration, end-tidal hydrogen concentration, end-tidal nitric oxide concentration, or end-tidal nitrogen dioxide concentration.

Another embodiment of the present invention provides an end-tidal gas monitoring apparatus for monitoring hydrogen sulfide gas in the exhaled breath of a mammal comprising a gas conduit configured for fluid communication with the exhaled breath of a mammal; a diverter valve in fluid communication with the gas conduit, wherein the diverter valve controls exhaled breath flow to a hydrogen sulfide gas sensor downstream of the diverter valve; a CO₂ sensor upstream of the diverter valve to denote the beginning and end of exhalation cycle in communication with a controller which determines end-tidal gas levels in the exhaled breath of a mammal to determine when the diverter valve should direct end-tidal gas flow to the gas sensor; and a recirculation loop downstream of the diverter valve to provide a continuous gas flow of end-tidal gas to the hydrogen sulfide gas sensor; and the hydrogen sulfide gas sensors being located in the recirculation loop.

Another embodiment of the present invention is directed to a method for monitoring a gas in exhaled breath of a mammal comprising collecting exhaled breath from a mammal; determining a predetermined level of end tidal CO₂ in the exhaled breath; directing gas flow to a gas sensor upon detection of the predetermined level of end tidal CO₂; optionally recirculating the exhaled gas to provide a continuous gas flow to the gas sensor; and determining a level of the exhaled gas in the exhaled breath. According to certain embodiments of the invention, the exhaled gas is end-tidal hydrogen sulfide, end-tidal carbon monoxide, end-tidal carbon dioxide, end-tidal hydrogen, end-tidal nitric oxide, or end-tidal nitrogen dioxide. According to certain embodiments of the invention, the method for monitoring a gas in exhaled breath of a mammal further comprises the step of indexing the exhaled gas to end tidal CO₂. According to certain embodiments of the invention, the exhaled gas is hydrogen sulfide, carbon monoxide, hydrogen, nitric oxide, or nitrogen dioxide. According to certain embodiments of the invention, the method for monitoring a gas in exhaled breath of a mammal further comprises collecting cumulative data on an amount of end-tidal gas exhaled by the mammal. According to certain other embodiments of the invention, the method for monitoring a gas in exhaled breath of a mammal further comprises sampling the exhaled breath of a mammal in a continuous manner. According to certain other embodiments of the invention, the method for monitoring a gas in exhaled breath of a mammal further comprises sampling the exhaled breath of a mammal in a periodic manner.

According to certain embodiments of the invention, the method for monitoring a gas in exhaled breath of a mammal further comprises the step of transmitting data resulting from gas analysis of the mammal's breath to a data processing unit. According to certain embodiments of the invention, the data processing unit includes a computer operably coupled to the one or more gas sensor component; a memory component operably coupled to the computer; and a database stored within the memory component.

Another embodiment of the present invention is directed to a method for monitoring a gas in exhaled breath of a mammal comprising: administering a therapeutic dose of a sulfide containing compound to the mammal to increase blood levels of sulfide; collecting exhaled breath from a mammal; determining a level of the exhaled gas in the exhaled breath; and comparing the level of the exhaled gas in the exhaled breath to a predetermined acceptable range of exhaled gas. According to certain embodiments of the invention, the method for monitoring a gas in exhaled breath of a mammal further comprises increasing the therapeutic dose of medicament if the measured level of the exhaled gas is below the predetermined acceptable range of exhaled gas; decreasing the therapeutic dose of medicament if the measured level of the exhaled gas is above the predetermined acceptable range of exhaled gas using predetermined levels of efficacy and safety to adjust dosage; or maintaining the therapeutic dose of medicament if the measured level of the exhaled gas falls within the predetermined acceptable range of exhaled gas.

### BRIEF DESCRIPTION OF DRAWINGS

FIG. 1 is a schematic representation of an end-tidal gas monitoring apparatus including gas conduit configured for fluid communication with the exhaled breath of a patient; a diverter valve in fluid communication with the gas conduit; a CO₂ sensor and one or more gas sensor according to one or more embodiment of the present invention.

FIG. 2 shows a graphical representation of a sampling of expired breath depicting the enrichment of the H₂S signal using the apparatus and method of the present invention. The graphical representation reflects a recording of data obtained from the apparatus using an artificial lung. The measured content of H₂S in exhaled breath is shown in the first channel (upper ⅓ of graph). The second channel (middle ⅓ of graph) is an indicator of actuation of the CO₂ based switch or diverter valve. The third channel (lower ⅓ of graph) is the oscillatory CO₂ pattern with each respiratory cycle. When the apparatus is first connected to the test lung (first vertical event mark), an oscillatory CO₂ pattern and an elevated exhaled H₂S is observed in comparison to the preceding time interval when the apparatus was disconnected and sampling room air. The second vertical event mark is change in computer command to the device allowing the CO₂ based switching of the diverter valve, whereupon a square wave signal is observed in the second channel, indicating switching of the diverter valve on/off. The introduction of switching the diverter valve enhances the capture of end-tidal breath, as the H₂S sensor is exposed to enriched end-tidal levels of H₂S, and as a result, the H₂S signal rises. The third vertical event mark is disconnecting the apparatus, at which point the CO₂ oscillations stop, the switching of the diverter valve stops, and the measured H₂S returns to reading of room air.

### DETAILED DESCRIPTION OF THE INVENTION

Before describing several exemplary embodiments of the invention, it is to be understood that the invention is not limited to the details of construction or method steps set forth in the following description. The invention is capable of other embodiments and of being practiced or being carried out in various ways.

The gas monitoring apparatus and method described herein provides the ability to monitor endogenous gas concentrations in a more cost effective and frequent manner. This method may be used to replace the invasive practice of drawing blood to measure concentration. Moreover, measurement of medications (and other substances) in exhaled breath may prove to be a major advance in monitoring a variety of drugs, compounds, naturally occurring metabolites, and molecules.

The present invention provides an apparatus and method for non-invasive monitoring of end-tidal gas concentrations in blood. More particularly, embodiments of the invention provide an apparatus and method for the detection, monitoring and trending of end-tidal gas concentrations, including hydrogen sulfide, carbon dioxide, carbon monoxide, nitric oxide and other respiratory gases, by utilizing one or more gas sensors to detect and measure concentration of such gaseous agents in exhaled breath.

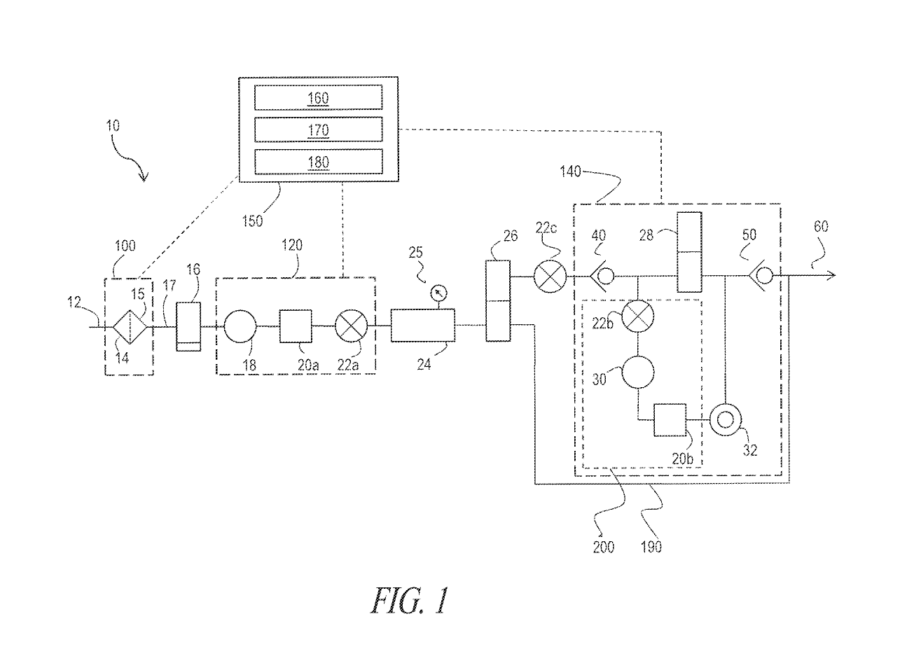

The end-tidal gas monitoring apparatus according to an embodiment of the present invention is illustrated in FIG. 1 and generally designated 10. As shown in FIG. 1, the end-tidal gas monitoring apparatus 10 includes a gas conduit and/or sample line 12, water filter and/or trap and/or particulate filter 14, zero valve 16, sample pump 18, one or more pneumatic filters (20a, 20b), one or more flow sensors (22a, 22b, 22c), CO₂ sensor 24, one or more diverter valve 26, bypass shutoff valve with the ambient port plugged 28, recirculation pump 30, and one or more gas sensor 32, recirculation loop inlet check valve 40, recirculation loop outlet check valve 50, and exit port 60. CO₂ sensor 24 may include one or more humidity, pressure, and/or temperature sensor(s) 25. Optionally, the apparatus includes a controller 150 and display (not shown) in communication with the apparatus to collect and output data collected by the apparatus 10. The controller can be on board the apparatus 10 or remotely located or hard wired to the apparatus as desired for particular applications.

A gas conduit 12 is disposed in the apparatus and fluidly connected to a mammal (not shown). In a specific embodiment, the mammal is a human. In another specific embodiment, the mammal is a human patient. In a specific embodiment of the present invention, the gas conduit is a sample line, which may be in the forum of a cannula or sample line. Gas conduit 12 has a substantially circular cross-section, or star-shaped to prevent kinking, and encloses a central flow pathway. The diameter of the gas conduit is chosen to provide the least appreciable resistance to the flow of the expired breath of the patient while still maintaining the integrity of the sample (i.e. little or no mixing of inhaled and exhaled gas sample).

The gas conduit 12 may be attached to a respiration collector (not shown) via a luer lock connector. In this specification, the term respiration collector refers to a component of, or accessory to, the flow module, through which the subject breathes. The respiration collector may comprise a mask, mouthpiece, face seal, nasal tubes, nasal cannula, nares spreader, trache tube, sample adapter, or some combination thereof. The respiration collector may include a mouthpiece, nosepiece or mask connected to the gas conduit 12 secured to the apparatus and adapted to be inserted into the mouth of a patient or over the nose and mouth of a patient, respectively for interfacing a patient to readily transmit the exhaled breath into the apparatus 10. In use, the respiration collector may be grasped in the hand of a user or the mask is brought into contact with the user's face so as to surround their mouth and nose. With the mask in contact with their face, the user breathes normally through the gas monitoring apparatus for a period of time.

A side-stream gas sample from a patient may be drawn from the sample line or gas conduit 12 attached to a breathing mask sample port, or a side stream sample adapter attached to a mask port or inserted into a mechanical ventilation breathing circuit between the patient-Y and the tracheal tube, or mask. The side-stream sample can also be drawn from a nasal cannula. The cannula may have multiple lumens where the other lumens are used to simultaneously deliver oxygen or other gasses, or are used to sample for other gases.

As shown in FIG. 1, the gas conduit 12 may be fluidly connected to a water management system 100 of the apparatus. The water management system 100 includes a water filter and/or trap and/or particulate filter 14 and an optional level sensor 15. The water filter and/or trap and/or particulate filter 14 may be of any suitable type for medical applications, including, but not limited to granular activated filters, metallic alloy filters, microporous filters, carbon block resin filters and ultrafiltration membranes. The optional level sensor 15 can be any suitable type sensor, including, but not limited to pulse-wave ultrasonic sensors, magnetic and mechanical float sensors, pneumatic sensors, conductive sensors, capacitive sensors, and optical sensors, an example being an Honeywell LLE series sensor. One or more water filter(s) and/or trap(s) and/or particulate filter(s) 14 may be disposed in the apparatus upstream of specific components to prevent contamination of these components. As shown in FIG. 1, in one embodiment of the present invention, a water filter and/or trap and/or particulate filter 14 is disposed downstream from the gas conduit 12 and upstream from a zero valve 16. The water management system 100 may monitor the water level sensor and alert the user when the water level is above a predetermined threshold so that the user can take appropriate action to empty or replace the container.

The water management system 100 of the apparatus may be connected via manifold or tubing 17, which may be Teflon lined, to a zero valve 16. In one embodiment of the present invention, the zero valve 16 may, for example, be a Magnum solenoid valve manufactured by Hargraves Technology Corporation, Morrisville, N.C. In one embodiment, as shown in FIG. 1, the zero valve 26 is a three-way valve. The zero valve 16 may be used to sample room air for calibration. The zero valve 16 may also be used to test for a blocked gas conduit 12 by checking if flow resumes when sampling air from the room environment versus sampling expired air from a patient via sample line or gas conduit 12.

Zero valve 16 is connected to a flow control system 120 via manifold or tubing 17. The flow control system 120 as shown includes a sample pump 18, a pneumatic filter 20a and a flow sensor 22a, all connected via manifold or tubing 17, along with the circuitry and microprocessor to execute a feed-back control loop to ensure that the sample pump 18 samples at a constant rate, typically in the range of 100 to 250 ml/min. The sample pump 18 can be any suitable pump which can be used for fluidly transmitting intake gases through the apparatus 10. Pneumatic filter 20a, as described in the present specification, is used to reduce pneumatic (or pressure) noise detected by the flow sensor 22a such that the flow control system 120 can function properly. The pneumatic filter 20 may be a resistor, a small added capacitative volume, a laminar flow element or some combination thereof. The pneumatic filter 20 is connected via manifold or tubing to a flow sensor 22 located downstream from pneumatic filter 20. Flow sensor 22 which may be used in embodiments of the present invention include: hot cable anemometers and other thermal methods, ultrasonic sensors (e.g. using the transit times of ultrasonic pulses having a component of direction parallel to the flow pathway, sing-around sensor systems, and ultrasonic Doppler sensors detecting frequency changes in ultrasound as it propagates through a gas), differential pressure sensors (such as a pneumotach), turbines, pitot tubes, vortex shedding sensors (e.g. detecting vortices shed by an element in the flow path), and mass flow sensors (22a, 22b, 22c). In a specific embodiment of the present invention, the flow sensor 22 is a hot surface anemometer or microbridge mass airflow sensor, such as a Honeywell AWM Series. Such microbridge mass airflow sensor use thin film temperature sensitive resistors.

The flow control system 120 is connected via manifold or tubing to a CO₂ sensor 24. The signal from the CO₂ sensor 24 may be utilized to indirectly measure CO₂, O₂, and respiration rate of the patient. CO₂ sensor 24 signal may be processed by the system controller (150) to provide breath-by-breath readings for end-tidal CO₂, and respiratory rate (breaths/minute). The signal from CO₂ sensor 24 may be automatically processed and adjusted for humidity, barometric pressure, and temperature of the gas sample. Adjustable alarms may be provided to monitor the level of CO₂ and respiratory rate. The alarms may be audible and or visual alarms or other suitable alarms to warn the patient or medical personnel of a condition that requires attention. In one embodiment of the present invention, the CO₂ sensor 24 measures CO₂ with a temperature-controlled miniature infrared analyzer cell; O₂ may also be measured with a paramagnetic sensor (not shown).

As shown in FIG. 1, in one embodiment of present invention, CO₂ sensor 24 is connected via a low volume connection to diverter valve 26, located downstream from the CO₂ sensor 24. In one embodiment as shown in FIG. 1, the diverter valve 26 is a three-way valve. A suitable diverter valve can be diverter valves available from Hargraves Technology Corporation, Morrisville, N.C.

In one embodiment, CO₂ sensor 24 is used to detect the starting and completion of exhalation. The gas sample is pumped through CO₂ sensor 24, where the beginning and end of a patient's exhalation phase can be detected with about a real-time signal response. During inhalation, the CO₂ signal is near 0%. As the patient begins to exhale, the CO₂ signal rises quickly. When the CO₂ signal exceeds a predetermined threshold, exhalation is determined to have started. When the CO₂ signal drops below a predetermined threshold, exhalation is determined to have ended. The predetermined thresholds may be different for the start and end of exhalation, and may change on a breath to breath basis or in real-time. Additional parameters may be utilized, such as minimum duration, to determine the start and end of an exhalation cycle.

It is contemplated that most side-stream infrared CO₂ sensors with a fast (for example, <30 ms) response time can be used in the present invention. One such CO₂ sensor a non-dispersive infrared CO₂ sensor, for example, a TreyMed Comet Sensor available from TreyMed, Inc. of Sussex, Wis.

In one embodiment of the present invention, a system controller 150 in electrical communication with the CO₂ sensor 24 analyzes the data stream coming from it. The communication between the controller 150 and components of the apparatus 10 can be by hard wired or wireless connections. The controller 150, which generally includes a central processing unit (CPU) 160, support circuits 170 and memory 180. The CPU 160 may be one of any form of computer processor that can be used in an industrial, consumer, or medical setting for processing sensor data and for executing control algorithms, various actions and sub-processors. The memory 180, or computer-readable medium, may be one or more of readily available memory such as random access memory (RAM), read only memory (ROM), flash, floppy disk, hard disk, or any other form of digital storage, local or remote, and is typically coupled to the CPU 160. The support circuits 170 are coupled to the CPU 160 for supporting the controller 150 in a conventional manner. These circuits include cache, power supplies, clock circuits, input/output circuitry, analog to digital converters, digital to analog converters, signal processors, valve control circuitry, pump control circuitry, subsystems, and the like. Where a display is included in the apparatus, the CPU also may be in communication with the display.

When end-tidal CO₂ is detected, the controller 150 controls the diverter valve 26 based on a predetermined algorithm calculating CO₂ thresholds, to divert the sample gas stream toward the gas sensor, thus exposing an electrochemical cell gas sensor located in the recirculation loop downstream only to end-tidal gas from a patient. The gas sensor may also be of another type, for example, a solid state or chemical luminescent, or infrared sensor.

In a specific embodiment, samples are taken of “end-tidal H₂S” which reflects the H₂S concentration in the lung. The end-tidal samples are then correlated with blood concentration of the gas using standard techniques or predetermined algorithms via a microprocessor in communication with the apparatus. In one embodiment of present invention, end tidal samples are used to compute a blood concentration of hydrogen sulfide based on the measured H₂S concentration in exhaled air and knowledge of the partial pressure of H₂S in context of other gasses in exhaled air, the volume of air exhaled, the rate of equilibration for H₂S gas between blood in pulmonary capillaries and air in the alveolar space and the solubility of H₂S gas in blood. In a specific embodiment, the gas sensor is a hydrogen sulfide sensor, preferably capable of detecting hydrogen sulfide in a sample in the range of 0-5000 ppb.

A diverter valve 26 is mounted upstream of both the recirculation loop 140, and the bypass pathway 190, which vents the sample to exhaust (into the room) when the controller 150 detects that the patient is not exhaling end-tidal gas. As illustrated in FIG. 1, one embodiment of the apparatus has a diverter valve 26 comprising a three way valve that opens into a pathway that is in fluid communication with the recirculation loop 140 containing gas sensor 32.

The exhaled gas proceeds from the diverter valve 26 to a flow sensor 22c and inlet check valve 40 and then into the recirculation loop, entering flow sensor 22b located downstream from the diverter valve 26. The flow sensor 22 is a conventional and/or miniaturized flow measuring sensor. One example of such a sensor is a hot surface anemometer, which is available from Honeywell. Other flow measuring sensors may be used in the apparatus as the application requires.

As shown in FIG. 1, in one embodiment of the present invention, more than one flow sensors may be used in the apparatus 10. Flow sensors 22a and 22b are primary flow sensor for the sample pump feedback control loop. Redundant components such as flow sensor 22c, along with additional valves 16 and 28 allow for automatic detection and diagnosis of device failure conditions while also providing a means for calibration. Primary flow sensor 22a and 22b can be cross-checked against the flow sensor 22c when the diverter valve 26 is in a “switched” state, meaning that it is diverting flow into the recirculation loop 140. Mismatch of flow between any one primary flow sensor 22a or 22b and redundant flow sensor 22c may indicate a leak or a problem with one of the flow sensors. The flow sensor 22c located downstream from the diverter valve 26 can also be used to test the function of the diverter valve 26.

In one embodiment of the present invention, a 3-way bypass shutoff valve 28, having a plugged port to ambient environment forces all gas flow into the recirculation loop which allows for cross check of the flow sensors 22a, 22b, and 22c, when the recirculation pump 30 is turned off. Flow sensor 22a, 22b, or 22c mismatch indicates problem with one of the three flow sensors or a leak. In other words, bypass shutoff valve 28 allows for comparison of all of the flow sensors 22a, 22b and 22c located in the apparatus.

The flow sensors 22a, 22b, and 22c may be in communication with a controller 150 so that any flow measured by the sensors is input into to the controller 150. The controller 150 may be in communication via electrical wiring or other communication means with a flow sensor 22.

In one embodiment of the present invention, the controller 150 processes signals provided by gas sensor 32, flow sensors (22a, 22b and 22c), and CO2 sensor to determine gas concentration and flow parameters, and, optionally, includes a memory to store the gas concentration or flow information or data. In one embodiment, the controller 150 manipulates the data provided by gas sensor 32, flow sensors (22a, 22b and 22c), and CO2 sensor to determined hydrogen sulfide concentration.

The flow sensor 22b is fluidly connected to a recirculation loop 140. In certain embodiments, the recirculation loop is a cylindrical reservoir having an inlet port for the influx of gas, such as breath, and an outlet port for the exhaust of breath. The exhaled gas proceeds from flow sensor 22b through the remainder of the recirculation loop, and may exit though outlet check valve 50 when new sample flow enters the recirculation loop. As shown in FIG. 1, the recirculation loop 140 may include one or more flow sensors 22b, recirculation pump 30, one or more pneumatic filters 20 and one or more gas sensor 32 each connected via tubing or manifold pathway.

As shown in FIG. 1, the recirculation loop is in flow communication with a recirculation pump 30. Recirculation pump 30 maintains a constant flow rate though a feedback control loop which executes on controller 150 utilizes flow sensor 22b as an input signal.

In operation, the sample of end-tidal breath, is pushed into recirculation loop 140 via sample pump 18 when the diverter valve 26 is in the “switched” state. Within the recirculation loop the end-tidal gas sample is transported by means of a recirculation pump 30 into the vicinity of the gas sensor. The gas sensor is in flow communication with the end-tidal breath of the patient.

Suitable recirculation pumps 30 include, but are not limited to, a fan, or an air pump. The recirculation loop or sensor may be heated to achieve an optimal or known gas sensing environment. The gas sensor is chosen from known materials designed for the purpose of measuring exhaled gases, vapors, such as, but not limited to hydrogen sulfide, carbon monoxide, and nitric oxide.

When a new sample of end-tidal gas is introduced into the recirculation loop, previously recirculating gas and or excess gas within the loop is exhausted though outlet check valve 50 and then finally though exhaust port 60.

Expired respiratory components which may be detected and/or analyzed using embodiments according to the present invention include one or more of the following: oxygen, carbon dioxide, carbon monoxide, hydrogen, nitric oxide, organic compounds such as volatile organic compounds (including ketones (such as acetone), aldehydes (such as acetaldehyde), alkanes (such as ethane and pentane)), nitrogen containing compounds such as ammonia, sulfur containing compounds (such as hydrogen sulfide), and hydrogen. In a specific embodiment of the present invention, the gas sensor may be a hydrogen sulfide sensor, oxygen sensor, carbon dioxide sensor, or carbon monoxide sensor. In a specific embodiment, gas sensor 32 is a H₂S or CO Fuel Cell sensor.

In a specific embodiment of the present invention, the hydrogen sulfide concentration of the exhalation flow is measured. While presently measured in an electrochemical cell, hydrogen sulfide may also be measured by alternate means such as gas chromatography or by utilizing the spectral properties of hydrogen sulfide gas (absorption of ultraviolet light).

Another specific embodiment of the present invention relates to a method to continuously monitor, in real time, the measurement of exhaled H₂S concentration as measured by an electrochemical cell gas sensor. Certain electrochemical cell gas sensors are excellent for detecting low parts-per-billion concentrations. Electrochemical cell sensors rely on an irreversible chemical reaction to measure. They contain an electrolyte that reacts with a specific gas, producing an output signal that is proportional to the amount of gas present. In a specific embodiment of the present invention, the electrochemical cell sensors used is for gases such as carbon monoxide, hydrogen sulfide, carbon dioxide, and/or nitric oxide.

However, electrochemical cells typically exhibit a very long response time to produce a signal. Therefore, in one embodiment of the present invention, a gas from the patient's nose and/or mouth is continually sampled.

Some electrochemical sensors require a constant flow of gas over the sensing surface. Because apparatus 10 introduces new exhaled gas samples to the sensor intermittently (during the exhalation only), the sensor may reside in a gas recirculation loop 140. The apparatus further includes a recirculation flow controller 200 containing flow sensor 22b, pump 30, and filter 20b, to provide a constant flow of gas over the sensing surface. The gas recirculation pump may be located within a recirculation loop or volume chamber.

The gas sensor 32 resides in the gas recirculation loop downstream of the recirculation pump 30 and pneumatic filter, as shown in FIG. 1. In one embodiment, the gas sensor 32 is a hydrogen sulfide sensor. The position of the sensor within the recirculation loop is also important, as the gas flow rate through the sensor or across the sensing surface must be constant.

According to one or more embodiments, the total volume of the sample in the recirculation loop is about 5 to 10 ml of volume. The total volume of the sample in the apparatus 10 can vary depending on how much of the end-tidal sample you want to “capture” in the recirculation loop. For example, if a patient is breathing at 12 breaths/minute, I:E ratio of 1:2, and the sample flow rate is 250 ml/min, approximately 14 mL of incoming sample flow per breath will be exhaled gas, a portion of which is end-tidal exhalation gas.

The total volume of the sample in the recirculation loop may be adjustable, along with the flow rate of the gas recirculation pump 30. Each time an exhalation occurs and a new gas sample is directed toward the gas sensor 32, the gas sample residing from the previous exhalation, along with any excess gas volume, is exhausted though a outlet check-valve 50 and exhaust port 60, into the room.

Real-time software algorithms running on a controller 150 control the main sample pump 18, recirculation sample pump 30, diverter valve 26. These algorithms also monitor the CO₂ sensor at a high sampling rate and determine when to acquire data from the gas sensor, e.g. H₂S electrochemical cell. The data acquired from the cell may be run though signal processing algorithms to provide a smooth signal that filters out noise, as well as, to detect peaks.

The end-tidal gas travels towards the gas sensor 32 located in the recirculation loop 140. When the end of the exhalation or end-tidal phase is detected, the diverter valve 26 is switched by the controller 150 such that the gas sample bypasses 140 the electrochemical cell gas sensor 32 via bypass pathway 190 and is exhausted outside the device through the exhaust port 60.

The apparatus may further comprise a system controller 150 adapted to interpret signals from sensors and transducers, circuitry to provide zeroing and calibration of the sensors and transducers, and circuitry to provide further processing of signals sent to the computation module (such as an analog to digital circuit, signal averaging, or noise reduction circuitry) and an electrical connector transmitting signals therefrom to a computation module.

### Software

In operation, the system controller 150 enables data collection and feedback from the respective systems such as water management system 100, flow control system 120, recirculation loop 140 and the subcomponents of these systems to optimize performance of the apparatus 10. In one or more embodiments, the apparatus is capable of displaying values or waveforms on a user-interface screen, such as H₂S, end-tidal H₂S, CO₂, end-tidal CO₂, and respiratory rate. Software routines, when executed by the CPU, and when in combination with input output circuitry, transform the CPU into a specific purpose computer (controller) 150. The software routines may also be stored and/or executed by a second controller (not shown) that is located remotely from the apparatus 10.

A software application program can be provided, executable by the CPU, to process input signals from sensors to calculate flow rates, flow volumes, oxygen consumption, carbon dioxide production, other metabolic parameters, respiratory frequency, end tidal nitric oxide, end tidal hydrogen sulfide, end tidal oxygen, end tidal carbon dioxide, end tidal nitric oxide, peak flow, minute volume, respiratory quotient (RQ), ventilatory equivalent (VEQ), or other respiratory parameters.

In one embodiment of the present invention, the end-tidal gas concentration monitoring apparatus may be used as analytical drug assay to measure, display and save, in real-time, a patient's end-tidal hydrogen sulfide concentration during the administration of sulfide-containing and sulfide-releasing compounds. A sulfide-containing compound is defined as a compound containing sulfur in its −2 valence state, either as H₂S or as a salt thereof (e.g., NaHS, Na₂S, etc.) that may be conveniently administered to patients. A sulfide-releasing compound is defined as a compound that may release sulfur in its −2 valence state, either as H₂S or as a salt thereof (e.g., NaHS, Na₂S, etc.) that may be conveniently administered to patients.

It is contemplated that the data accumulated via the end-tidal gas concentration monitoring apparatus of the present invention may be used to guide future research and clinical studies, and assist in future safety decisions made by medical personnel or governmental regulatory agencies, e.g., U.S. Food and Drug Administration.

It is contemplated that an embodiment of the present invention may serve as a safety monitor, providing audio-visual warning to a medical practitioner or clinician when one or more of a patient's end-tidal gas concentrations, e.g., hydrogen sulfide, drifts outside of alarm thresholds set by the medical practitioner or clinician. Alarms are set to notify the clinician when breaths are not detected as well as when measured ETH₂S exceeds a set alarm threshold.

The device is capable of logging data in real-time while measuring from a patient. This data is logged to the device's internal memory, or to an external device such as a flash drive. The data may also be exported so that it can be collected by an external device via serial, USB, Ethernet, or other communication means. The data includes snapshots of what is being displayed on the user-interface screen, as well as real-time data from the sensors (processed or raw), alarm information, the current operation mode, calibration information, or other internal or diagnostic information. In accordance with embodiments of the present invention, data from a particular patient are stored so that multiple samples over an extended period of time may be taken.

The collected CO₂ data may be processed to calculate and output respiratory parameters of the respiratory system such as respiratory rate, end tidal CO₂, and to determine when the diverter valve should be in the “switched” mode. The sampled end-tidal breath is processed by hydrogen sulfide sensors to calculate the concentration of hydrogen sulfide contained therein.

In one or more embodiments of the present invention, high and low alarms for specific concentrations of measured gas concentration may be set by the user, and the settings may be stored in non-volatile memory so they do not have to be reset the next time apparatus 10 is used. In one embodiment, a controller 150 may be connected to an external computer via a serial port which provides all the measurements in a simple format for collection by the external computer. The serial port may provide simple ASCII formatted data that can be received using any communications software, and easily imported into a spreadsheet for calculation.

In specific embodiments, alerts may be generated for end tidal partial pressure, concentration, or derived index of H₂S, CO₂, and/or respiration rate. Minimum and maximum threshold values for each of these parameters are set by a user or are predetermined. As the end tidal partial pressure, concentration, or derived index of H₂S, CO₂, and/or respiration rate are determined, they are compared to the set thresholds. Sampled values which fall below their respective minimum threshold or exceed their respective maximum threshold trigger an alert. Similarly, the monitoring of and alerts for other parameters are also within the scope of the present invention.

### Sampling Modes

Sampling is defined as any means of bringing gas into contact with the end tidal monitoring apparatus 10.

The end-tidal gas monitoring apparatus is capable of running in multiple modes: continuous sampling or end-tidal “switching” sampling mode. When calibrating the apparatus, continuous sampling is used.

### Continuous Sampling

The device may also operate in a continuous mode when sampling from the patient, while end-tidal exhalation time is integrated using the CO₂ sensor. In continuous mode all of the sample flow, rather than just the end-tidal portion, from the patient is diverted toward the recirculation loop 140 in fluid communication with the gas sensor 32, e.g., a H₂S gas sensor. The resulting endogenous gas reading, e.g., H₂S concentration, can be corrected based on the calculated I:E ratio to provide peak exhaled or end tidal H₂S using a software algorithm.

When breaths are not detected for a period of time (as determined by a software algorithm monitoring the CO₂ sensor) a software algorithm may determine that the gas sample chamber or recirculation loop should be flushed out, at which point the device automatically enters a continuous sampling mode. Once adequate CO₂ is detected a software algorithm will determine that the patient is once again breathing and the device may automatically revert to the “switched” end-tidal sampling mode. When operating in continuous mode the recirculation loop is not necessary.

It has been determined that blood-based assay approaches are not feasible for measuring hydrogen sulfide. H₂S sensors are slow-responding electrochemical sensors that consume H₂S gas molecules continuously. This invention utilizes the patient's CO₂ signal to determine when exhalation is occurring, allowing for selective enrichment of the exhaled gas around the H₂S electrochemical sensor.

Recirculation gas flow through or around the surface of the H₂S sensor satisfies the flow rate requirements of the electrochemical sensor. In addition, proper placement of the sensor within the recirculation loop ensures the flow rate though or across the surface of the electrochemical sensor remains constant.

When no exhaled breaths are detected for a pre-determined period of time, e.g. 30 seconds, or the system is no longer connected to the patient e.g when the apparatus is booting up, the recirculation loop is flushed out by having the sensor exposed to ambient gas from the room.

### Calibration

The end tidal gas monitoring apparatus 10 should be calibrated as required, which may be done by sampling a gas of known composition into the end tidal gas monitoring apparatus 10. A gas-filled canister may be provided for this purpose. It is also important to purge the sampling device after use to discharge excess moisture or other components. Purging could be done, for example, by sampling dry medical air or room air into the end tidal gas monitoring apparatus 10. In such a system, the two functions of calibration and purging may thereby be performed in a single step. Alternatively, the calibration gas and the purging gas may be different, and the two functions performed in separate steps. Certain types of analyzers are more stable and require less calibration than others. An algorithm running on the controller 150 may monitor the status of apparatus 10 to determine when it needs calibrating

According to one or more embodiments, prior to patient use, the end tidal monitoring apparatus, and in particular, the gas sensor 32, is calibrated. This is accomplished by sampling a gas of known composition into the device. A canister of such gas is provided for this purpose. The apparatus 10 may also sample from the room to obtain a 0 ppb source for the calibration.

In specific embodiments, there is a 2-point calibration for apparatus 10. The first point is the zero, the sensor output at which the gas concentration is 0 ppb H₂S and 0% CO₂. The second point is the span, which is ideally obtained at a point above the highest expected measurement from the patient. An exemplary span point is at 5000 ppb H₂S and 12% CO₂. The sensor output is linear between the two points, or fit to a curve that is known or measured. The device is calibrated at regular time intervals. The device may also attempt to detect when a calibration is needed, for example, when no breaths are detected and the sensor is measuring above or below 0 ppb, the device may prompt the user to perform a calibration.

Some or all aspects of the calibration may be automated, while some aspects of the calibration may require the user to take action such as connect H₂S or CO₂ calibration gas. The device has additional zero valves 16 that can be automatically actuated by the software algorithms that control calibration. The execution of these calibration algorithms may be triggered automatically.

The sample flow sensor 22a may be calibrated using an external flow sensor, measuring inlet or outlet flow. The recirculation flow sensor 22b may be calibrated by switching diverter valve 26 to bypass mode, and by removing the plug from bypass shutoff valve 28 so that when bypass shutoff valve 28 is switched to bypass mode, the recirculation pump 30 then pulls in ambient air though bypass shutoff valve 28. Upstream of the ambient port (when unplugged) of valve 28 an external flow sensor can be used as a reference to calibrate flow sensor 22b.

After calibration, a sample of expired breath is taken. Finally, after patient use, the system samples room air to purge the pneumatic pathways to prevent contaminants from building up in the apparatus 10. This may also be accomplished by providing a gas of known composition for sampling such as pure dry air, and may be combined with a calibration step.

One or more embodiments of the present invention provides a method for monitoring exhaled hydrogen sulfide levels in patients before, during and after an administration of therapeutic sulfide-releasing or sulfide-containing compounds is provided. Sulfide is defined as sulfur in its −2 valence state, either as H₂S or as a salt thereof (e.g., NaHS, Na₂S, etc.) that may be conveniently administered to patients. One or more embodiments of the present invention provides a method for the measurement of exhaled hydrogen sulfide which may serve as a potential safety marker for future clinical trials involving sulfide and sulfide-releasing compounds.

### Use of Apparatus for H₂S Gas Monitoring

A specific application of the apparatus shown in FIG. 1 can be for monitoring H2S gas. As with the above described methods, the apparatus receives exhaled breath of a subject and the apparatus measures the concentration of one or more components in the exhaled breath, including H₂S. As noted above, it is desirable to calibrate the apparatus prior to taking a sample of expired breath.

The patient is instructed to perform normal tidal breathing which is sampled via sample line or respiration collector for several breaths. Continuous sampling over multiple breaths collected by the side stream method is preferable. In one embodiment of the present invention, samples are collected through a sample line or gas conduit 12 which may be connected to an adapter at the proximal end of a respiration collector and drawn through Teflon-lined tubing to the apparatus 10, having one or more gas sensors 32.

The expired breath travels through the water filter and/or trap and/or particulate filter 14 and zero valve 16 towards the sample pump 18. In operation, the sample pump 18 causes the gas sample from the patient (not shown) to travel therethrough in downstream direction towards the CO₂ sensor 24. During the pumping, the flow within the apparatus is monitored with the flow sensors (22a, 22b, 22c). The exhaled breath travels into the recirculation loop 140, having a gas sensor 32 via the diverter valve 26. The gas sample is pumped through the CO₂ sensor 24, where the beginning and end of a patients' exhalation phase can be detected with near a real-time signal response. The controller 150 communicates with the CO₂ sensor 24 and analyzes the data stream coming from it. During inhalation, the CO₂ signal at the CO₂ sensor 24 is near 0%. As the patient begins to exhale, the CO₂ signal rises quickly. When the CO₂ signal exceeds a predetermined threshold, end-tidal exhalation is determined to have started. To begin the end-tidal sampling process when end-tidal CO₂ is detected based on a predetermined algorithm calculating and monitoring CO₂, the controller 150 transmits a signal to open the diverter valve 26 into the recirculation loop to divert the sample gas stream toward the gas sensor, thus exposing the electrochemical cell gas sensor 32, e.g., H₂S sensor, only to the end-tidal gas. The end-tidal sample then recirculates though or over the H₂S sensor within recirculation loop 140. Recirculation pump 30, located within the recirculation loop, provides a constant flow of end-tidal gas past the H₂S sensor.

When the CO₂ signal drops below a predetermined threshold exhalation is determined to have ended, the controller 150 transmits a signal to switch the diverter valve 26 such that the recirculation loop is bypassed via bypass pathway 190 and the sample gas stream is exhausted toward the room environment through exhaust port 60. Each time a new end-tidal sample is detected and diverted into the recirculation loop 140, the previous end tidal sample exists the recirculation loop 140, along with excess new sample gas volume, though the outlet check valve 50, though the exhaust port 60, into the room environment.

An analog-to-digital converter may be used to measure and process data from the gas sensor, as well as archive data to a memory source. Software within a controller 150 may be used to process data further to generate summary parameters and values to quantify exhaled sulfide measurements.

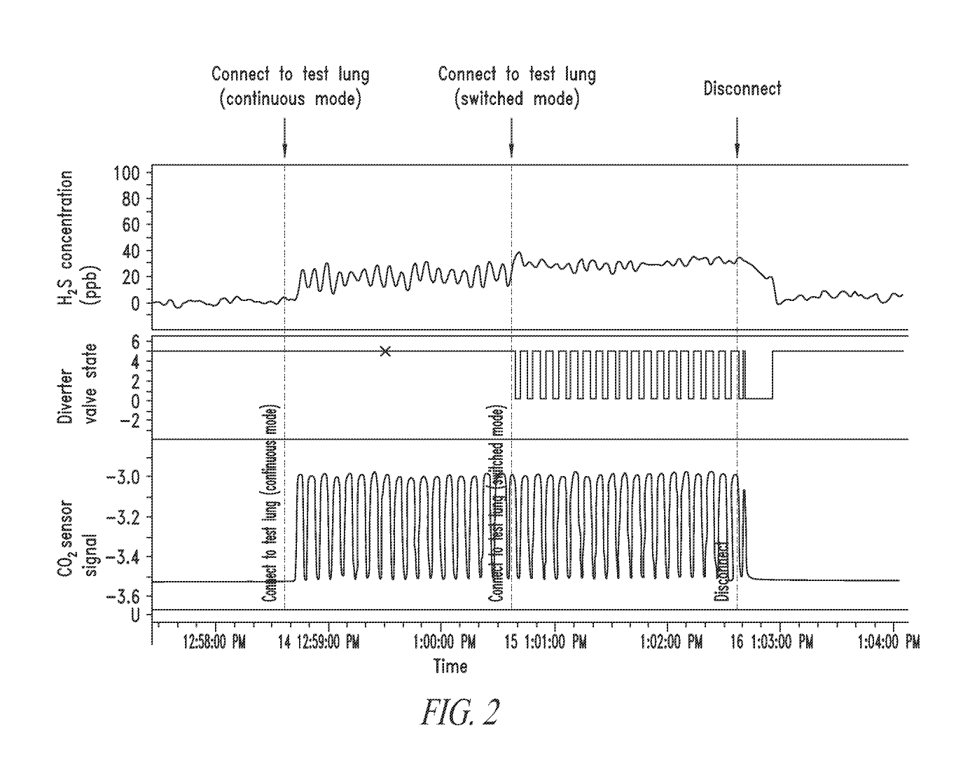

FIG. 2 shows a graphical representation of a sampling of expired breath depicting the enrichment of the H₂S signal using the apparatus and method of the present invention. The graphical representation reflects a recording of data obtained from the apparatus using an artificial lung. The measured content of H₂S in exhaled breath is shown in the first channel (upper ⅓ of graph). The second channel (middle ⅓ of graph) is an indicator of actuation of the CO₂ based switch. The third channel (lower ⅓ of graph) is the oscillatory CO₂ pattern with each respiratory cycle. When the apparatus is first connected to the test lung (first vertical event mark), an oscillatory CO₂ pattern and an elevated exhaled H₂S is observed in comparison to the preceeding time interval when the apparatus was disconnected and sampling room air. The second vertical event mark is change in computer command to the device allowing the CO₂ based switching, whereupon a square wave signal is observed in the second channel, indicating switching on/off. The introduction of switching enhances the capture of end-tidal breath and as a result, the H₂S signal rises. The third vertical event mark is disconnecting the apparatus, at which point the CO₂ oscillations stop, the switching stops and the measured H₂S returns to reading of room air. The top trace is the H₂S signal, the middle trace is the on/off toggling of the 3-way valve, and the bottom trace is the CO₂ signal. The first half of the data was collected with the device in continuous mode (note the 3-way valve position is held constant). The second half of the data was collected in switching mode, note the toggling of the diverter valve 26 in synchrony with the CO₂ signal, and the enrichment of the H₂S signal.

In one embodiment of the present invention, apparatus 10 is used to measure the concentration of H₂S gas in exhaled air, wherein the measurement of exhaled sulfide may subsequently be used by a medical practitioner in the diagnosis of an illness. In another embodiment, apparatus 10 is used to detect alterations in endogenous sulfide levels which may be indicative of presence of a disease state or progression of disease.

In one embodiment of the present invention, apparatus 10 is used to measure the concentration of exhaled H₂S gas in an individual, wherein the measurement of exhaled sulfide may subsequently be used by a medical practitioner to monitor a response to the administration of a medicament designed to increase blood levels of sulfide. In a specific embodiment, apparatus 10 is used to measure and monitor the concentration of exhaled H₂S gas in an individual being administered parenteral sulfide therapy.

Apparatus 10 may be used in combination with the administration of a medicament which is designed to increase blood levels of sulfide where the knowledge of exhaled sulfide guides the administration of a medicament in order to avoid administration of an amount which is excessive and potentially unsafe.

Apparatus 10 may be used in combination with the administration of a medicament which is designed to increase blood levels of sulfide where the knowledge of exhaled sulfide levels guides the administration and adjustment of dosage of the medicament to achieve a safe therapeutic amount of the medicament. For example, the therapeutic dose of medicament may be increased if the measured level of the exhaled gas is below the predetermined acceptable range of exhaled gas; the therapeutic dose of medicament may be decreased if the measured level of the exhaled gas is above the predetermined acceptable range of exhaled gas; or the therapeutic dose of medicament will be maintained if the measured level of the exhaled gas falls within the predetermined acceptable range of exhaled gas.

“Therapeutically effective amount” refers to that amount of a compound of the invention which, when administered to a mammal, preferably a human, is sufficient to effect treatment, as defined below, of a disease or condition in the mammal, preferably a human. The amount of a compound of the invention which constitutes a “therapeutically effective amount” will vary depending on the compound, the condition and its severity, the manner of administration, and the age of the mammal to be treated, but can be determined routinely by one of ordinary skill in the art having regard to his own knowledge and to this disclosure.

“Treating” or “treatment” as used herein covers the treatment of the disease or condition of interest in a mammal, preferably a human, having the disease or condition of interest, and includes: (i) preventing the disease or condition from occurring in a mammal, in particular, when such mammal is predisposed to the condition but has not yet been diagnosed as having it; (ii) inhibiting the disease or condition, i.e., arresting its development; (iii) relieving the disease or condition, i.e., causing regression of the disease or condition; or (iv) relieving the symptoms resulting from the disease or condition. As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

In one embodiment, apparatus 10 may be configured such that output information from apparatus 10 can become input commands for communication with an infusion pump to administer a medicament which is designed to increase blood levels of sulfide. In a specific embodiment, apparatus 10 controls the administration of a medicament utilizing a feedback loop designed to maintain safe and efficacious administration of medicament.

In one embodiment, apparatus 10 may be used to measure end-tidal gas concentrations in the exhaled breath of human patients subjected to increasing doses of medications in human safety and tolerability studies, e.g., as required by the U.S. Food and

### Drug Administration.

In another embodiment, apparatus 10 may be used to measure H₂S concentrations in the exhaled breath of human patients subjected to increasing doses sodium sulfide in human phase I safety and tolerability studies.

In another embodiment, apparatus 10 is capable of detecting 1-5000 ppb hydrogen sulfide in exhaled breath.

In another embodiment, a predetermined range of 1-50 ppb hydrogen sulfide in exhaled breath may be established in apparatus 10 as the quantity normally present in exhaled breath of healthy human subjects.

In another embodiment, a predetermined range of 100-800 ppb hydrogen sulfide in exhaled breath may be established in apparatus 10 as the quantity associated with efficacious outcomes in treatment of diseases.

In another embodiment, a user programmable visible or audible alarm is set in apparatus 10 when the detected amount of hydrogen sulfide in exhaled breath equals or exceeds a value considered as potentially unsafe, e.g. 1000 ppm.

In another embodiment, apparatus 10 is capable of computing blood or plasma levels of hydrogen sulfide based on the observed exhaled fraction and other physiologic parameters (respiratory rate, body temperature).

Reference throughout this specification to “one embodiment,” “certain embodiments,” “one or more embodiments” or “an embodiment” means that a particular feature, structure, material, or characteristic described in connection with the embodiment is included in at least one embodiment of the invention. Thus, the appearances of the phrases such as “in one or more embodiments,” “in certain embodiments,” “in one embodiment” or “in an embodiment” in various places throughout this specification are not necessarily referring to the same embodiment of the invention. Furthermore, the particular features, structures, materials, or characteristics may be combined in any suitable manner in one or more embodiments.

Although the invention herein has been described with reference to particular embodiments, it is to be understood that these embodiments are merely illustrative of the principles and applications of the present invention. It will be apparent to those skilled in the art that various modifications and variations can be made to the method and apparatus of the present invention without departing from the spirit and scope of the invention. Thus, it is intended that the present invention include modifications and variations that are within the scope of the appended claims and their equivalents.

What is claimed is:

1. An end-tidal gas monitoring apparatus for monitoring gas in the exhaled breath of a mammal comprising:
a gas conduit configured for fluid communication with the exhaled breath of a mammal;
a diverter valve in fluid communication with the gas conduit, wherein the diverter valve controls gas flow to a gas sensor downstream of the diverter valve;
a CO₂ sensor upstream of the diverter valve in communication with a controller which determines CO₂ levels in the exhaled breath of a mammal to determine when the diverter valve should direct gas flow to the gas sensor; and
a recirculation loop downstream of the diverter valve to provide a continuous gas flow to the gas sensor.

2. The end-tidal gas monitoring apparatus of claim 1, wherein the gas sensor is a hydrogen sulfide gas sensor, carbon monoxide gas sensor, carbon dioxide gas sensor, hydrogen gas sensor, nitric oxide gas sensor, or nitrogen dioxide gas sensor.

3. The apparatus of claim 1 further comprising: computer operably coupled to the gas sensor component; a memory component operably coupled to the computer; a database stored within the memory component.

4. The apparatus of claim 3, wherein the computer is configured to calculate and collect cumulative data on an amount of exhaled gas by the mammal.

5. The apparatus of claim 4, wherein the exhaled gas is end-tidal hydrogen sulfide, end-tidal carbon monoxide, end-tidal carbon dioxide, end-tidal hydrogen, end-tidal nitric oxide, or end-tidal nitrogen dioxide.

6. The apparatus of claim 4, wherein the computer is capable of providing information that alerts a user of the computer of a significant deviation of exhaled gas concentrations from predetermined exhaled gas levels.

7. The apparatus of claim 6, wherein the exhaled gas concentration is end-tidal hydrogen sulfide concentration, end-tidal carbon monoxide concentration, end-tidal carbon dioxide concentration, end-tidal hydrogen concentration, end-tidal nitric oxide concentration, or end-tidal nitrogen dioxide concentration.

8. An end-tidal gas monitoring apparatus for monitoring hydrogen sulfide gas in the exhaled breath of a mammal comprising:
a gas conduit configured for fluid communication with the exhaled breath of a mammal;
a diverter valve in fluid communication with the gas conduit, wherein the diverter valve controls exhaled breath flow to a hydrogen sulfide gas sensor downstream of the diverter valve;
a CO₂ sensor upstream of the diverter valve to denote the beginning and end of exhalation cycle in communication with a controller which determines end-tidal gas levels in the exhaled breath of a mammal to determine when the diverter valve should direct end-tidal gas flow to the gas sensor; and
a recirculation loop downstream of the diverter valve to provide a continuous gas flow of end-tidal gas to the hydrogen sulfide gas sensor; and
the hydrogen sulfide gas sensors being located in the recirculation loop.

9. A method for monitoring a gas in exhaled breath of a mammal comprising:
collecting exhaled breath from a mammal;
determining a predetermined level of end tidal CO_{2 in} the exhaled breath;
directing gas flow to a gas sensor upon detection of the predetermined level of end tidal CO₂;
optionally recirculating the exhaled gas to provide a continuous gas flow to the gas sensor; and
determining a level of the exhaled gas in the exhaled breath.

10. The method of claim 9 wherein the exhaled gas is end-tidal hydrogen sulfide, end-tidal carbon monoxide, end-tidal carbon dioxide, end-tidal hydrogen, end-tidal nitric oxide, or end-tidal nitrogen dioxide.

11. The method of claim 9 further comprising the step of indexing the exhaled gas to end tidal CO₂.

12. The method of claim 11 wherein the exhaled gas is hydrogen sulfide, carbon monoxide, hydrogen, nitric oxide, or nitrogen dioxide.

13. The method of claim 9 further comprising collecting cumulative data on an amount of end-tidal gas exhaled by the mammal.

14. The method of claim 9 further comprising sampling the exhaled breath of a mammal in a continuous manner.

15. The method of claim 9 further comprising sampling the exhaled breath of a mammal in a periodic manner.

16. The method of claim 9 further comprising the step of transmitting data resulting from gas analysis of the mammal's breath to a data processing unit.

17. The method of claim 9 wherein the data processing unit includes a computer operably coupled to the one or more gas sensor component; a memory component operably coupled to the computer; a database stored within the memory component.

18. A method for monitoring a gas in exhaled breath of a mammal comprising:
administering a therapeutic dose of a sulfide containing compound to the mammal to increase blood levels of sulfide;
collecting exhaled breath from a mammal;
determining a level of the exhaled gas in the exhaled breath; and
comparing the level of the exhaled gas in the exhaled breath to a predetermined acceptable range of exhaled gas.

19. The method of claim 18 further comprising: a) increasing the therapeutic dose of medicament if the measured level of the exhaled gas is below the predetermined acceptable range of exhaled gas; b) decreasing the therapeutic dose of medicament if the measured level of the exhaled gas is above the predetermined acceptable range of exhaled gas using predetermined levels of efficacy and safety to adjust dosage; or maintaining the therapeutic dose of medicament if the measured level of the exhaled gas falls within the predetermined acceptable range of exhaled gas.
